(19) United States
(12) Patent Application Publication
(10) Pub. No.: US 2006/0084901 A1
(43) Pub. Date: Apr. 20, 2006

(54) Dressing holder and a combination of a foot bed and such a dressing holder

(75) Inventors: Leif Lindh, Danderyd (SE); Kjell Lindh, Lidingo (SE); Peter Kohler, Stocksund (SE); Pelle Netz, Stockholm (SE)

Correspondence Address: JACOBSON HOLMAN PLLC, 400 SEVENTH STREET N.W., SUITE 600, WASHINGTON, DC, 20004, US

(73) Assignee: DANDERYDS BIOTECH INNOVATION AB, Danderyd (SE)

(21) Appl. No.: 11/220,409

(22) Filed: Sep. 7, 2005

Related U.S. Application Data
(63) Continuation of application No. 10/476,487, filed on Nov. 7, 2003, now abandoned.

(30) Foreign Application Priority Data
May 16, 2001 (SE) 0101725-0

(51) Int. Cl. A61F 15/00; A61F 13/00

(52) U.S. Cl. 602/41

(57) ABSTRACT

A dressing support that includes a flexible, resilient spacing sheet, which is stuck firmly to the patient and has an opening for receiving an ulcerated area. A compress is applied in the opening. A foot bed includes a recess which is complementary to the support and which receives the support when the foot rests on the foot bed, the support being stuck to the bottom or plantar surface of the foot.

The present invention relates to a dressing support of the kind defined in the preamble of the independent claim directed to the dressing support.

The invention also relates to a foot bed in combination with the inventive dressing support, in accordance with the preamble of the independent claim directed to the combination.

Diabetics are prone to skin sores, particularly on the underside or soles of their feet. Such sores do not heal easily, due to the poor circulation and the impaired activity of the sufferer. According to one conventional method, sores of this nature are healed by providing a relatively spacious shoe and placing in the shoe a foot bed which is generally complementary to the sole of the patient's foot. There is provided in the foot bed at the location of the sore a recess which can also accommodate a dressing placed over the sore on the patient's foot. The bandage/dressing and the sore are relieved of pressure, when the patient wears the correct treatment shoe. The patient is able to move freely while wearing the shoe, since only those parts of the foot that withstand load are subjected to pressure against the foot bed, whereas the dressing and the sore are subjected to no load at all. The dressing may have the form of a compress on which there may be applied some appropriate medicament and which is applied to the patient's foot over the sore and changed when necessary.

One problem with this solution is that patients are often of the opinion that a treatment shoe fitted with a foot bed is not particularly attractive aesthetically, and consequently prefer not to wear the shoe, and fail to take the trouble and the time to put the shoe on when indoors or when walking short distances or making short errands. Another problem is that on special occasions patients wear more attractive shoes which are not fitted with a pressure-relieving foot bed. As a result, the sore is subjected to pressure via the compress, therewith seriously impairing the healing process or delaying said process. The problem is accentuated by the fact that diabetics normally have impaired tactility on the soles of their feet and consequently are unable to feel troublesome pain caused by pressure on the sore.

When changing a compress or dressing, another problem often resides in the failure to give the new compress the same size, thickness and positioning on the foot as the preceding compress, from which the foot bed is designed or modelled. There is consequently a risk that the sore will be subjected to load, since the shape and size of the compress do not coincide with the original form of the foot bed.

A further problem is that even when the compress is positioned correctly initially, this position can be disturbed when the foot is inserted into or removed from the foot bed placed in the treatment shoe.

Accordingly, one object of the invention is to eliminate these problems, either completely or partially.

A further object of the invention is to provide a dressing support that covers either part of the foot or the whole of the foot and that can be firmly adhered to the patient's foot and functions to relieve pressure on the sore even in the absence of a foot bed, and that the specially produced compress/dressing that fits in the support will always have the same size and shape. When changing the dressing support, it shall be replaced with a support of the same size and in accordance with indications placed precisely in accordance with the former compress/dressing so as to fit in the foot bed.

A further object of the invention is to provide a foot bed which is adapted to a patient's foot and which includes a recess corresponding to the dressing support.

These objects are achieved either totally or partially by means of the present invention.

The invention is defined in respective accompanying independent claims.

Further embodiments of the invention are set forth in the accompanying dependent claims.

A central part of the invention relates to a dressing support that can be used to relieve pressure on a sore or on a sensitive skin surface of a patient and also to firmly secure a dressing on the patient and to enable a compress/dressing to be changed while the dressing support is seated firmly on the patient, i.e. stuck to the patient's skin.

The dressing support can be applied to the underside of a patient's foot such as to prevent direct contact between the underlying foot supporting surface and a sore or a sensitive area on the underside of the foot. The dressing support suitably has the form of a resilient pressure-distributing sheet or strip of material that includes a through-penetrating opening in which a compress/dressing can be placed without serious danger of loading the surface that shall be relieved of pressure should the patient support his/her weight on the underlying surface via the dressing support. The foot bed can probably be excluded in certain instances, e.g. when the dressing support covers the whole of the underside of a patient's foot and includes one or more through-penetrating compress/dressing-accommodating openings.

It will be understood, however, that the inventive dressing support also finds use in the treatment of other skin areas or skin surfaces of a patient, where there is a risk of sores forming in local areas as a result of pressure, for instance bedsores, e.g. above the sacrum.

The dressing support is intended for one-time use only and basically has the form of sheet material (in different sizes and with different sized holes for compresses/dressings) e.g. a flexible pressure-distributing material (for example, gelatine material, polyurethane, EVA or like materials). The sheet is provided with a skin-friendly adhesive and is intended and designed to remain stuck to the patient over a continuous period of time of up to several weeks. If the support is removed, it cannot be replaced since otherwise the support might be wrongly positioned. The dressing support includes a through-penetrating opening for accommodating a compress/dressing and forms a spacing means which prevents the sore beneath the compress in the support opening from being subjected to pressure/load. A medicament can be applied locally to the compress/the dressing when necessary. The dressing constantly has the same shape and size and can be replaced via the opening in the support with the support stuck firmly to the patient.

The opening receiving the compress/dressing can be sealed with adhesive tape, for instance. The outer surface of the sheet-like support is preferably slippery so as to reduce the risk of the support being displaced by forces acting in the plane of the support. The side of the support that lies proximal to the underlying foot-supporting surface, e.g. the floor, may be provided with a stiff layer that distributes the load or pressure points and therewith reduces the risk of local pressures on the patient's foot sore. This layer will preferably be flexible so as to adapt to the shape of the surface area of the patient on which the dressing support shall be stuck.

By producing the dressing support in several standard designs, and by providing the support with graphic markings and constructing the support so that it can readily be brought to the correct size by cutting pieces from the dressing support, for instance whilst guided by said graphic markings, a fresh dressing support can be applied in a precisely correct position when changing the support, with the aid of markings provided on the body part to this end, and the size of the opening can be adjusted.

When the dressing support has been securely adhered to the underside of a patient's foot with the opening in the support positioned over a sore or a tactile area on the underside of the foot, a foot bed can be cast or moulded on a conventional way, with the foot and the applied dressing support as a model.

When the patient with the dressing support applied to his/her foot inserts his/her foot into the treatment shoe, the foot bed and the dressing support will together provide a comfortable support surface for the whole of the foot in the treatment shoe, with the exception of the opening in the dressing support.

In those cases when the dressing support covers the whole of the foot with pressure-relieving openings for sores or tactile skin areas, the adaptation of a normal shoe may suffice.

When the patient removes the treatment shoe, the dressing support carrying the dressing will remain on his/her foot, wherewith the dressing support will prevent direct contact between the walking surface and the sore as the patient walks without the treatment shoe/foot bed.

The fact that the exposed side of the dressing support is slippery reduces the risk of the support being disturbed on the foot of the wearer as he/she inserts her foot in or on to the foot bed in the treatment shoe.

When the foot to which the support is applied shall be inserted into a sock or stocking, the slippery outer surface of the support affords the advantage of reducing the risk of displacement of the support relative to the foot and also facilitates insertion of the foot carrying the support into the sock.

As will be understood, the foot bed may be formed on the basis of the foot to which a dressing support has been applied being clothed with a sock/stocking.

The slippery/smooth outer layer of the support may include one or more secondary openings, and the support may include a soft elastic rubber-like layer nearest the outer layer that includes said secondary openings. As load is exerted on the dressing support, parts of the rubber layer will penetrate plastically out through the secondary openings and therewith form anti-slip elements, which are particularly useful when a patient that has a dressing support applied to his/her foot walks on a slippery floor without wearing a sock or stocking.

The invention will now be described by way of example with reference to the accompanying drawing.

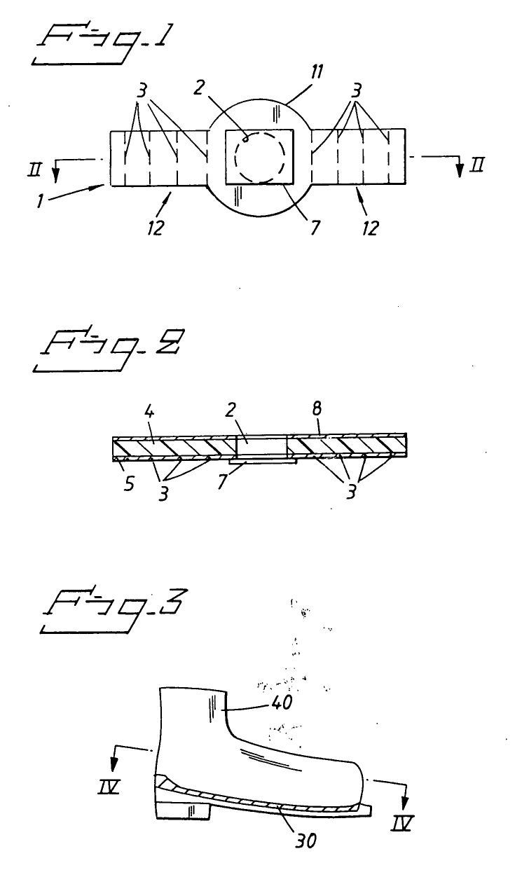

FIG. 1 is a plan view of one example of an inventive dressing support.

FIG. 2 is a sectional view taken on the line I-I in FIG. 2.

FIG. 3 is a schematic vertical sectioned view of a treatment shoe fitted with a foot bed.

FIG. 4 is a view of the upper side of the foot bed.

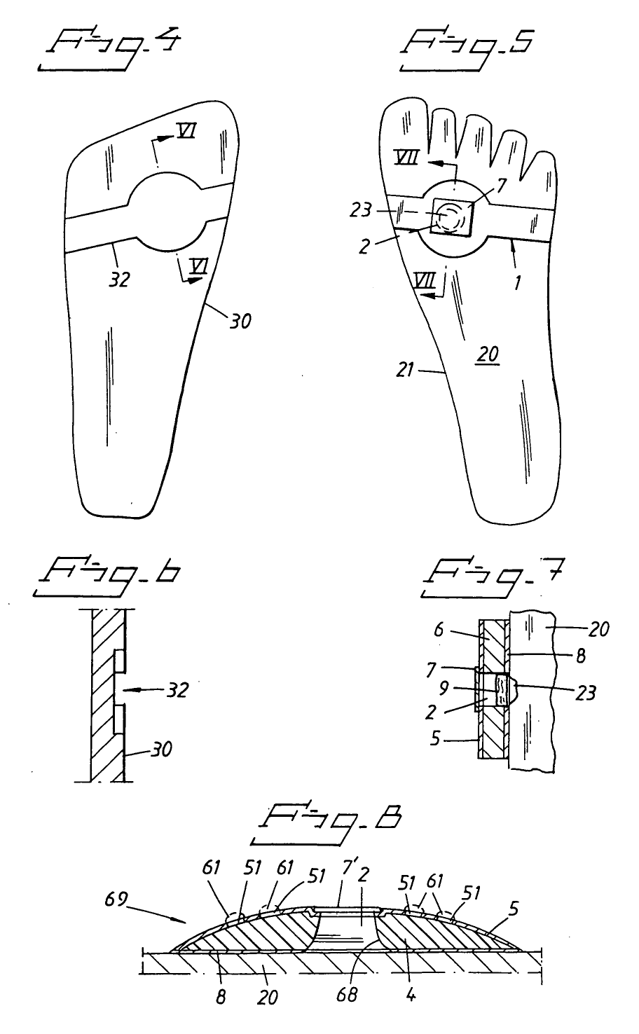

FIG. 5 is a plan view of the underside of a patient's foot to which a dressing support has been applied.

FIG. 6 is a schematic section view taken on the line VI-VI in FIG. 4.

FIG. 7 is a schematic section view taken on the line VII-VII in FIG. 5.

FIG. 8 is a schematic cross-sectional view of a dressing support.

FIG. 1 illustrates a standardised sheet-like dressing support 1, which includes an opening 2 whose area corresponds at most to one-third of the area of the support 1. The support 1 also includes graphic markings 3 in the form of indications that facilitate cutting of the support to a desired size.

A support 1 intended for a patient's foot may include a roundish central part 11 having a diameter of, e.g., 5 cm and including a central opening 2 having a diameter of 2.5 cm. Strip-like parts 12 connect with diametrically opposed parts of the round portion 11. The strip parts 12 have a length of about 7 cm. The side 4 of the support that is intended to lie proximal to the patient, may be formed from a load-distributing, soft plastic sheet which is firmly fastened to adhesive tape 8 or the like used to secure the support 1 to the patient. The exposed side of the support 1 is formed from a load-distributing, relatively rigidly flexible sheet 5 of material, which has a slippery exposed outer surface. A compress (9), possibly provided with a local medicament, is placed in the space defined by the opening 2. Tear-off adhesive tape 7 or the like may be adhered to the sheet 5 over the openings (2).

A dressing support 1 is placed on the underside 20 of a patient's foot 21 (FIG. 5) in a position in which the opening 2 is in alignment with a sore 23 on the underside 20 of the foot. The strip parts 12 are cut so that the support 1 will not extend beyond the plane contour of the underside of the foot, and the support 1 is now stuck firmly to the underside 20 of the foot, for instance with double-sided adhesive tape.

A foot bed 30 (FIG. 2) is now produced in a conventional fashion, by using as a model the underside 20 of the foot 21 with the support firmly affixed thereto. The actual foot bed 30 includes a recess 32 whose plane contour corresponds to the plane contour of the fitted support 1, and has a depth corresponding to the thickness of said support. The underside of the foot bed 30 is given a form that will enable it to be correctly fitted in a treatment shoe (FIG. 3), which is preferably designed to facilitate the insertion and removal of a patient's foot to which a support 1 has been applied.

Because the underside 5 of the support 1 is slippery, there is less danger of the support 1 being displaced relative to the foot 21 as a result of friction against, e.g., the foot bed 30 when putting on the shoe 40.

The adhesive tape 7 (or alternatively a cover 7′ (FIG. 8)), which preferably also has a slippery outer surface, can be readily pealed or torn off to enable the compress in the opening 2 in the support part 11 to be replaced whilst the support 1 remains firmly stuck to the patient's foot 21.

The support 1 includes an intermediate layer 6 of flexible material, for instance a formable plastic layer.

A patient who has removed his/her foot from the shoe 40 with the support 1 attached is able to walk on a floor surface with less danger of the sore 23 being subjected to pressure loads from the floor, since the support 1 functions as a spacing means.

The inventive dressing support finds general use as a means for preventing direct contact between a tactile surface area of a patient's skin or a skin sore and an underlying supportive surface, and can, of course, be given other configurations than that shown in FIG. 1. The embodiment illustrated in FIG. 1 is particularly suitable for protecting foot sores or sensitive areas of a patient's skin and can be provided in a few standard designs which together enable the majority types of foot sores of typical sizes and in typical places to be treated. It will be understood that several supports 1 can be applied to a patient's foot. Alternatively, a complete sole that includes several openings.

For example, a dressing support may have more than one opening 2 and can, of course, also retain its basic function, namely that of forming a spacing means which surrounds a sore or a tactile area and which provides favourable transfer of load between an adjacent underlying support surface and an area on the patient that surrounds the sore or the tactile area.

An inventive dressing support can thus be used beneficially by, for instance, rheumatics or neuropatients for reasons other than those associated with diabetes, for instance the presence of a sensitive growth on the underside of the foot and that can be supported with the aid of a dressing support, possibly without the dressing, wherewith the foot and the dressing support affixed thereto can be received in a correspondingly shaped bed in a shoe in the same way as that described above.

In one embodiment of the invention, the outer edge of the dressing support 1 may be chamfered, bevelled 69 or thinned so that the outer edge part of the applied dressing support will taper gently so as to lie flush with the patient's skin, i.e. in the absence of any protrusion.

The defining edge of the opening 2 may also be bevelled 68, said bevel 68 preferably lying nearest the patient so as to provide pressure relief in the area around the edge of the opening. As an alternative to the tape 7, there can be used a cover 7′ which can be fitted loosely into the opening in the proximity of the outer surface of the sheet-like. The cover 7′ is suitably sunken in the opening, so as to reduce the risk of the cover transferring pressure loads to the patient and/or to a dressing located in the opening.

The outer layer 5, which is relative rigid, has a number of openings 51 that enable the soft elastic material in the layer 4 to protrude out through said openings 51 so as to form friction studs 61 when the support 1 is compressed between the patient's foot and an underlying surface, such as a floor, for example. The studs 61 reduce the risk of the support sliding on a slippery undersurface when subjected to load.

1-12. (canceled)

13. The combination of a dressing support and a foot bed comprising:
a dressing support including a flexible sheet having a first main surface for adhesive adherence to skin on a patient's sole, and a smooth or slippery second main surface opposite to said first main surface, said sheet having a through-penetrating opening and, when applied to the skin, having a thickness between said first and second main surfaces that forms a spacing element between a surface area of the patient's skin surrounded by said opening and an underlying footbed support surface which subjects the patient to load via said sheet; and
a footbed molded or cast with the patient's sole having the dressing support firmly affixed thereto as a model so that said molded or cast footbed has an individual topography corresponding thereto, the molded or cast footbed having a recess in an upper side thereof whose plane contour corresponds to a plane contour of the dressing support, said recess including a sunken area that is in alignment with an ulcerous area or other pain-sensitive area on said sole, said through-penetrating opening of the dressing support being accommodated within the sunken area and a remainder of said dressing support being accommodated within the recess of the molded or cast footbed when the patient's foot, with the dressing support firmly adhered thereto, rests on the foot bed.

14. The combination according to claim 13, further comprising a compress placed in said opening.

15. The combination according to claim 13, wherein said thickness of said dressing support diminishes from an area adjacent said opening toward an outer edge part of said dressing support through a tapering thereof such that said outer edge part lies flush with the skin.

16. The combination according to claim 13, wherein the recess is generally complementary to the dressing support and further accommodates a stocking or sock placed on the foot over the dressing support.

17. The combination according to claim 13, wherein the sheet has a plane form of a circular part having radially and outwardly projecting strip parts, said opening being disposed centrally in said circular part and being accommodated in said sunken area, said recess further including elongated portions that accommodate said projecting strip parts.

18. The combination according to claim 13, wherein said sheet includes a flexible elastomeric layer between said first and second main surfaces, said second main surface including a load-distributing outer layer.

19. The combination according to claim 18, wherein said outer layer includes one or more secondary openings through which said elastomeric layer elastically penetrates when said sheet is subjected to load so as to form exposed anti-slip studs projecting from said second main surface.

20. The combination according to claim 13, wherein a defining edge of said sheet opening is bevelled on the first main surface.

21. The combination according to claim 13, wherein said sheet has an associated cover that can be loosely applied over the opening, said cover being sunken into an outer part of the opening.

22. The combination according to claim 13, wherein said sheet includes markings which provide references for cutting the sheet to obtain a desired shape and form.

23. The combination according to claim 22, wherein subsequent to cutting said sheet the sheet has a net area that corresponds to at least three times an area of the opening.

24. The combination according to claim 13, further comprising a compress placed in said opening.

25. A method of fitting a patient having an ulcerous area or a tactile area on the sole of a foot with a combination of a dressing holder and a foot bed comprising:
adhering a dressing support to the patient's sole, said dressing support including a flexible sheet having a first main surface for adhesive adherence to skin on the patient's sole, and a smooth or slippery second main surface opposite to said first main surface, said sheet having a through-penetrating opening and, when applied to the skin, having a thickness between said first and second main surfaces that forms a spacing element between a surface area of the patient's skin surrounded by said opening and an underlying footbed support surface which subjects the patient to load via said sheet; and
molding or casting a footbed with the patient's sole having the dressing support firmly affixed thereto as a model so that said molded or cast footbed has an individual topography corresponding thereto, the molded or cast footbed having a recess in an upper side thereof whose plane contour corresponds to a plane contour of the dressing support, said recess including a sunken area that is in alignment with an ulcerous area or other pain-sensitive area on said sole, said through-penetrating opening of the dressing support being accommodated within the sunken area and a remainder of said dressing support being accommodated within the recess of the molded or cast footbed when the patient's foot, with the dressing support firmly adhered thereto, rests on the foot bed.

26. The method as set forth in claim 25, further comprising before the step of adhering, the step of cutting the sheet along markings provided thereon to obtain a desired shape.

27. The method as set forth in claim 26, wherein said step of cutting includes cutting said sheet to have a net area that corresponds to at least three times an area of the opening.

28. The method as set forth in claim 26, wherein said step of cutting includes cutting said sheet to have a plane form of a circular part having radially and outwardly projecting strip parts, said opening being disposed centrally in said circular part.

29. The method as set forth in claim 28, wherein said step of molding or casting the footbed includes forming the recess such that said circular part is accommodated in said sunken area and said recess includes elongated portions that accommodate said projecting strip parts.

30. The method as set forth in claim 25, further comprising the step of placing a compress in said opening.
